(19) United States
(12) Patent Application Publication
(10) Pub. No.: US 2011/0300216 A1
(43) Pub. Date: Dec. 8, 2011

(54) Chewable, swallowable and effervescent solid dosage form for oral delivery of pharmaceutical actives

(76) Inventors: Eric R. First, Morristown, NJ (US); Ashish Bipin Patel, West Orange, NJ (US); Hung-Huar Tong, Basking Ridge, NJ (US)

(21) Appl. No.: 12/802,275

(22) Filed: Jun. 3, 2010

(51) Int. Cl. A61K 9/46; A61P 29/00; A61K 31/616

(52) U.S. Cl. 424/466; 514/165

(57) ABSTRACT

A tablet comprising: (a) an active ingredient; and (b) at least one member of an effervescent couple, wherein said tablet dissolves in water in not more than three minutes and in the mouth in not more than thirty seconds, and wherein said tablet is capable of acting as an effervescent tablet, a rapid-dissolve tablet or a rapidly disintegrating swallow tablet.

### BACKGROUND OF THE INVENTION

1. Field of the Invention

The invention relates to a solid-dose tablet useful for administering pharmaceuticals, over-the-counter medicines, vitamins and nutritional supplements. The tablet is formulated so that it can be administered by any conventional oral administration method such as dissolving in water, swallowed with water, or chewed without water.

2. Description of Related Art

Solid dosage forms for medicines have been known almost as long as medicines themselves. The problems with solid dosage forms have been known almost as long. Every solution to a problem with a solid dosage form seems to bring with it a new set of formulation challenges.

A tremendous improvement in dosage convenience and reliability came with the advent of compressed tablets. Patients no longer needed to worry about measuring doses accurately, and large scale pharmaceutical manufacturing operations could replace direct preparation by a local pharmacist. Unfortunately compressed tablets take longer to dissolve in the stomach and small intestine after administration than powders or other forms that are not compressed. This slower dissolution can delay the onset of the benefit of the medicine, which can be especially frustrating for administration of pain medications, where the need for rapid onset is acute.

One solution to the problem of slow dissolution came with the advent of effervescent tablets. Effervescent tablets usually come in the form of a compressed tablet that contains an edible acid and an edible base. These compounds are usually referred to as an effervescent couple, and evolve gas, usually carbon dioxide gas, when introduced to water. When the effervescent tablet is put in a glass of water, the neutralization reaction between the acid and the base liberates the gas, which makes the tablet fizz. The tablet quickly dissolves in the water, and the solid dosage form has been converted into a liquid solution or suspension that quickly enters the blood stream upon ingestion and is easier for the patient to swallow.

Formulators face many problems in preparing effervescent tablets that are acceptable for commercial applications. Upon reaction, the effervescent couple can leave a solid salt residue at the bottom of the glass that can lessen the appeal of the product to the consumer either through the salty taste or the visual appearance of the residue. After the reaction, the resulting solution can be cloudy, or the solution can have an unpleasant taste due to the taste of the medicine or to the salt formed by reaction of the effervescent couple.

Another problem with effervescent tablets, however, is that a glass of water is not always available for immediate use. As a result, formulators have looked to other approaches to the problem of slow dissolution of compressed tablets. These approaches include various forms of tablets that rapidly break down, either in the stomach or in the mouth. As the tablet rapidly falls apart, the greatly increased surface area of the solid material encourages more rapid dissolution into the stomach or mouth, and rapid absorbance in the body.

One such “quick release” tablet is described in U.S. Pat. No. 4,855,326 to Fuisz, issued Aug. 8, 1989. In the approach set forth in this patent, a medicine is incorporated into a matrix of melt spun sugar to form a tablet that quickly disintegrates in the mouth.

Another approach to rapid dissolution is to provide a mildly effervescent tablet that allows a tablet to dissolve in the mouth. This approach has not proven popular, except for a brief fad as a sugared candy, because the conflicting demands put on the effervescent couple are very difficult to resolve. Faster tablet disintegration comes from large amounts of the effervescent couple, but the increasing amounts of carbon dioxide that are generated in the mouth rapidly become unacceptable to the patient. Conversely, as less effervescent material is used, tablet disintegration slows, which can also make the tablet unacceptable for the patient, especially if the medicine has an unpleasant taste.

A tablet may be swallowed, either intact or in several pieces, and thereafter disintegrate in the stomach. Various methods have been suggested to prepare tablets that would be useful in implementing this approach, including the use of an effervescent couple in the tablet. The same problem of gas generation encountered in effervescent fast-dissolve tablets, however, is also encountered in so called “swallow” tablets that are designed to dissolve quickly in the stomach. Too little effervescence means slow disintegration and too much effervescence can have unacceptable consequences for the patient. One patent, U.S. Pat. No. 6,764,696 to Pather et al., issued Jul. 20, 2004, uses enteric coated effervescent materials to assist in increased absorbance of poorly absorbed medicines. Other approaches to effervescent control include hot-melt extrusion of the effervescent couple as described in U.S. Pat. No. 6,649,186 to Robinson et al. issued Nov. 18, 2003.

A final problem when designing tablets lies in the different patient preferences for the different approaches that may be adopted by the formulator. Some patients prefer effervescent tablets that are placed in a glass of water. Some prefer tablets that disintegrate in the mouth and some prefer tablets that are swallowed directly. The formulator responding to patient preferences has thus been required to develop many different formulations to meet these preferences. Providing different formulations requires separate manufacturing processes with corresponding costs and other issues. A single formulation that could be used as an effervescent tablet, a swallow tablet, and a chewable or quick dissolve tablet could provide significant cost savings for manufacturers and could provide significant benefits for consumers who would be able to choose the type of administration route desired for each tablet consumed.

### SUMMARY OF THE INVENTION

The principal object of the invention therefore is to provide a solid dosage form, such as a tablet, having a single formulation that is capable of being used as an effervescent tablet, a swallow tablet, and a chewable or rapid dissolve tablet.

Another object of the invention is to provide a solid dosage form, such as a tablet, capable of being used as an effervescent, swallow or rapid dissolve tablet that provides acceptable solution characteristics upon administration as an effervescent tablet, and acceptable organoleptic qualities when used as an effervescent, swallow or rapid dissolve tablet.

Additional objects and advantages of the invention will be set forth in part in the description that follows, and in part will be obvious from this description, or may be learned by practice of the invention. The objects and advantages of the invention may be realized and attained by means of the instrumentalities and combinations particularly pointed out in the appended claims.

To achieve the foregoing objects and in accordance with the purpose of the invention, as embodied and broadly described herein, the invention provides a tablet comprising an active ingredient, preferably with an optimized particle size, one or both members of an effervescent couple, disintegrants and other ingredients, such as excipients, sweeteners, flavors and taste masking agents, if desired.

### BRIEF DESCRIPTION OF THE FIGURES

FIG. 1 is a graph showing the average dissolution profile of an embodiment of the invention compared with two commercial formulations.

FIG. 2 is a graph showing the average dissolution profile of another embodiment of the invention compared with two commercial formulations.

FIG. 3 is a graph showing the average dissolution profile of a third embodiment of the invention compared with two commercial formulations.

FIG. 4 is a graph showing the average dissolution profile of a fourth embodiment of the invention compared with two commercial formulations.

### DETAILED DESCRIPTION OF THE PREFERRED EMBODIMENTS OF THE INVENTION

Reference will now be made in detail to the presently preferred embodiments of the invention.

The invention comprises an active ingredient, at least one member of an effervescent couple and, optionally, a disintegrant. Tablets made in accordance with the invention may be formulated with other ingredients well known in the art of tablet making, such as flow agents, binders, flavors, sweeteners, excipients, lubricants, tableting aids, fillers, surfactants, coloring agents, and other materials known in the art of tablet making.

The invention comprises at least one active ingredient. The active ingredient may be any medicine, whether available by prescription or over the counter, a vitamin, mineral or other nutritional supplement, or any therapeutic ingredient intended to benefit the patient that is able to be administered orally. Preferred active ingredients include pain relievers, agents for relieving cold or allergy symptoms, and agents for relief of gastrointestinal afflictions including indigestion and heartburn.

In one embodiment, the active ingredient of the invention is an acidic or basic compound capable of interacting with one member of an effervescent couple. With such active ingredients, the amount of one member of the effervescent couple can be reduced or even eliminated to reduce the size of the resulting tablet and to help control the effervescent reaction rate. One especially preferred active ingredient is aspirin, acetyl salicylic acid (“ASA”). In formulations containing aspirin, the acid component of the effervescent couple may be reduced or entirely eliminated because aspirin can act as an acid in effervescent tablets.

The active ingredient may comprise from about 1% to about 95% of the tablet of the invention. If the active ingredient does not interact with one member of the effervescent couple, thereby reducing or eliminating the need for the other member of the effervescent couple, then the active ingredient preferably comprises less than about 50% by weight of the tablet and more preferably less than about 20% of the tablet and most preferably less than about 10% of the tablet. If the active ingredient does replace some or all of one member of the effervescent couple, then the active ingredient may comprise from about 40% to about 95% of the tablet, more preferably from about 60% to about 90% by weight of the tablet and most preferably from about 70% to about 90% by weight of the tablet.

An effervescent couple includes an acid and a base that evolves a gas upon reaction. Each member of the effervescent couple in the invention is liberated when the tablet is brought into contact with water. Preferred acid and base combinations react with each other to produce carbon dioxide gas, which imparts carbonation to the aqueous solution when the tablet is used as an effervescent tablet and evolves gas bubbles when used as a swallow tablet or a rapid dissolve tablet. In the preferred embodiment set forth above, one component of the effervescent couple can be partially or completely replaced by an acidic or basic active ingredient. Preferred acids include edible acids known in the art, such as citric acid, ascorbic acid, malic acid, adipic acid, tartaric acid, and fumaric acid.

The base component of an effervescent couple preferably is capable of generating a gas upon reaction with either the acid member of the effervescent couple or with the active ingredient, if the active ingredient is acidic. Preferred bases include edible carbonate bases such as sodium bicarbonate, sodium carbonate, potassium carbonate, potassium bicarbonate, calcium carbonate, magnesium carbonate.

If the active ingredient is not acidic, the acid may comprise from about 5% by weight to about 50% by weight of the tablet, preferably from about 10% by weight to about 40% by weight of the tablet, and most preferably from about 12% to about 35% by weight of the tablet. If the active ingredient is acidic, the acid component of the effervescent couple may comprise from about 0% to about 30% by weight of the tablet, more preferably from about 0% to about 20% by weight of the tablet, and most preferably from out 0% to about 10% by weight of the tablet.

If the active ingredient is not basic, the base may comprise from about 5% by weight to about 50% by weight of the tablet, preferably from about 10% by weight to about 40% by weight of the tablet, and most preferably from about 12% to about 20% by weight of the tablet. If the active ingredient is basic, the acid component of the effervescent couple may comprise from about 0% to about 30% by weight of the tablet, more preferably from about 0% to about 20% by weight of the tablet, and most preferably from out 0% to about 10% by weight of the tablet.

The amount of the active ingredient in a tablet formulation is determined by the dosage needs of the patient being treated. Preferably the dosage to be administered is put into a single tablet, but the dosage may be split between two tablets or distributed among three or more tablets. Once the dosage level has been determined, the amount of acid, if any, and base, if any, is determined by evaluating the desired level of effervescence and the desired pH of the dissolved tablet. In a preferred embodiment of the invention, the amounts of acid and base (and active ingredient, if acidic or basic) are formulated to produce a pH of about seven upon completion of the effervescent reaction.

Tablets preferably have a hardness of from about 50 to about 150 newtons, preferably from about 75 to about 125 newtons, and more preferably about 100 newtons.

The tablet of the invention should dissolve in water in under three minutes, preferably with release of carbon dioxide gas. The tablet should also have adequate taste masking for chewing without water and should not overproduce carbon dioxide in the mouth. The tablet should also have enough integrity to last about thirty seconds in the mouth before it is swallowed with water. One preferred technique in preparation of the active ingredient is micronization. Micronization is known in the art and may be advantageously used with the active ingredient and one or both members of the effervescent couple.

Taste masking may be accomplished by adding a known taste-masking system, but, preferably, the tablet is formulated to reduce or eliminate the salt taste that can result when the tablet is consumed. The presence of a taste masking system can affect the clarity of the solution if the tablet is used as an effervescent tablet. A taste masking system can also precipitate and leave an undesirable residue at the bottom or rim of the container when the tablet is used as an effervescent tablet.

An important parameter in formulation is the balance between having a tablet that dissolves quickly and with adequate effervescence in water, soft enough to chew yet sturdy enough to swallow with a glass of water, and doesn't release too much carbon dioxide in the stomach when swallowed.

In one highly preferred embodiment of the invention, the effervescent material is combined with a disintegrant. Especially preferred is a class of compounds known as super-disintegrants. One especially preferred family of super-disintegrants is known by the commercial name Kollidon® disintegrants. This family comprises soluble and insoluble grades of polyvinylpyrrolidone of various molecular weights and particle sizes, a vinylpyrrolidone/vinyl acetate copolymer and a blend of polyvinyl acetate and polyvinylpyrrolidone. These materials are also known as povidone, crospovidone and copovidone. Preferred super-disintegrants include crospovidone: Kollidon CL (110-130 μm), CL-F (20-40 μm), CL-SF (10-30 μm), CL-M (3-10 μm), cellulose derivatives, croscarmellose sodium, and hydroxypropyl cellulose starch derivatives such as sodium starch glycoate.

One especially preferred member of the family is Kollidon® CL-SF. This material is identified as a crosslinked polyvinylpyrrolidone also called crospovidone, crospovidonum, insoluble polyvinylpyrrolidone, and crosslinked PVP. The material is identified as having CAS No. 9003-39-08. The main function this material is to act as a super-disintegrant in especially small tablets to fasten disintegration and dissolution. It has a pleasant mouth feel. Other super-disintegrants include croscarmelose, other forms of crospovidone, and sodium starch glycolate.

There is no universal ideal disintegrant, and the selection of the best disintegrant for each application must be determined on a case by case basis. As shown in the examples below, however, crosslinked polyvinylpyrrolidone is preferred for use in tablets of the invention containing standard doses of aspirin. The usual quantity of super-disintegrant used is preferably from about 0.5% to about 10% by weight of the tablet formulation. The presence of a super-disintegrant can have an adverse effect on transparency when the tablet is used as an effervescent tablet. More preferably, therefore, the super-disintegrant comprises from about 0.5% to about 1% by weight of the tablet.

Tablets of the invention are prepared using conventional tableting processes.

### Examples 1-8

Formulations in accordance with the invention were prepared by micronizing aspirin in an Airjet micronizer, to a particle size of d50 less than 30 microns as measured by particle size analyzer and blending the micronized aspirin with sodium carbonate and, optionally, a crosslinked polyvinylpyrrolidone, Kollidon® CL-SF (identified as “Kollidon” in the tables). Each ingredient is in the form of a powder and the powders were blended in a V-blender for about fifteen minutes. The blend was then subjected to roller compaction through a roller gap of 2.9 mm and at a vacuum pressure of 0.890 millibars. The roller compacter was rotated at 5.9 rpm; the screw feeder was set at 35 rpm and the granulator at 90 rpm. The hydraulic pressure was set at 20 bars. The blend was then formed into tablets that nominally contained either 325 mg aspirin or 500 mg aspirin. Table 1 shows the formulations of 325 mg aspirin tablets and Table 2 refers to 500 mg aspirin tablet formulations. The formed tablets were then weighed and the average tablet weight determined. In Example 1, only the aspirin was micronized. In example 2, both the aspirin and the carbonate were micronized separately. In example 3, the disintegrant was eliminated, which increase the transparency of the solution when the tablet was used as an effervescent tablet, as shown in Table 2. In example 4, the amount of carbonate was increased to accelerate the effervescent effect. The results are set forth in Table 1.

TABLE 1
Formulation of 325 mg Aspirin Tablets (Examples 1-4)

| | Ex. 1 | | Ex. 2 | | Ex. 3 | | Ex. 4 | |
|---|---|---|---|---|---|---|---|---|
| | Wt. % | Mg/tablet | Wt. % | Mg/tablet | Wt. % | Mg/tablet | Wt. % | Mg/tablet |
| Aspirin | 87.1 | 325.0 | 87.1 | 325.0 | 88.0 | 324.7 | 84.1 | 324.6 |
| Sodium | 11.9 | 44.4 | 11.9 | 44.4 | 12.0 | 44.3 | 14.9 | 57.5 |
| Carbonate | | | | | | | | |
| Kollidon | 1.0 | 3.7 | 1.0 | 3.7 | 0.0 | 0.0 | 1.0 | 3.86 |
| Total | 100.0 | 373.1 | 100.0 | 373.1 | 100.0 | 369.0 | 100.0 | 386.0 |

| | | | | |
|---|---|---|---|---|
| ID | 1019-JT-1 | 1019-JT-2 | 1019-JT-4 | 312-JT-2 |

TABLE 2
Formulation of 500 mg Aspirin Tablets (Examples 5-8)

| | Ex. 5 | | Ex. 6 | | Ex. 7 | | Ex. 8 | |
|---|---|---|---|---|---|---|---|---|
| | Wt. % | Mg/tablet | Wt. % | Mg/tablet | Wt. % | Mg/tablet | Wt. % | Mg/tablet |
| Aspirin | 87.1 | 500.0 | 87.1 | 500.0 | 88.0 | 500.0 | 84.1 | 500.0 |
| Sodium | 11.9 | 68.3 | 11.9 | 68.3 | 12.0 | 68.2 | 14.9 | 88.5 |
| Carbonate | | | | | | | | |
| Kollidon | 1.0 | 5.7 | 1.0 | 5.7 | 0.0 | 0.0 | 1.0 | 5.9 |
| Total | 100.0 | 574.0 | 100.0 | 574.0 | 100.0 | 568.2 | 100.0 | 594.4 |

| | | | | |
|---|---|---|---|---|
| ID | 1019-JT-1 | 1019-JT-2 | 1019-JT-4 | 312-JT-2 |

### Examples 9-12

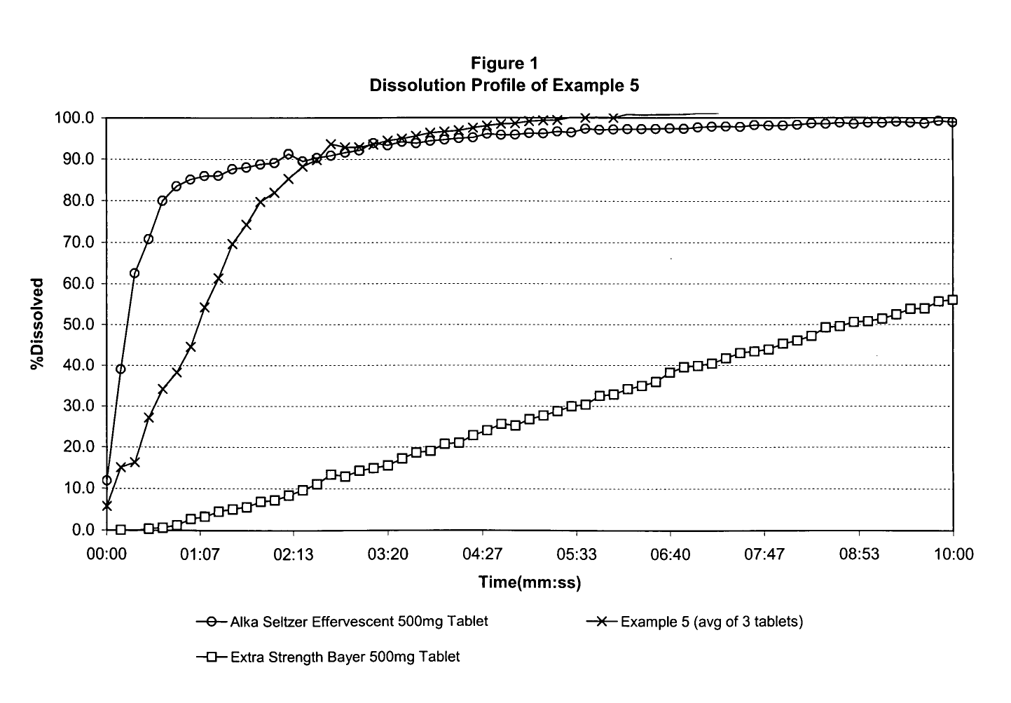

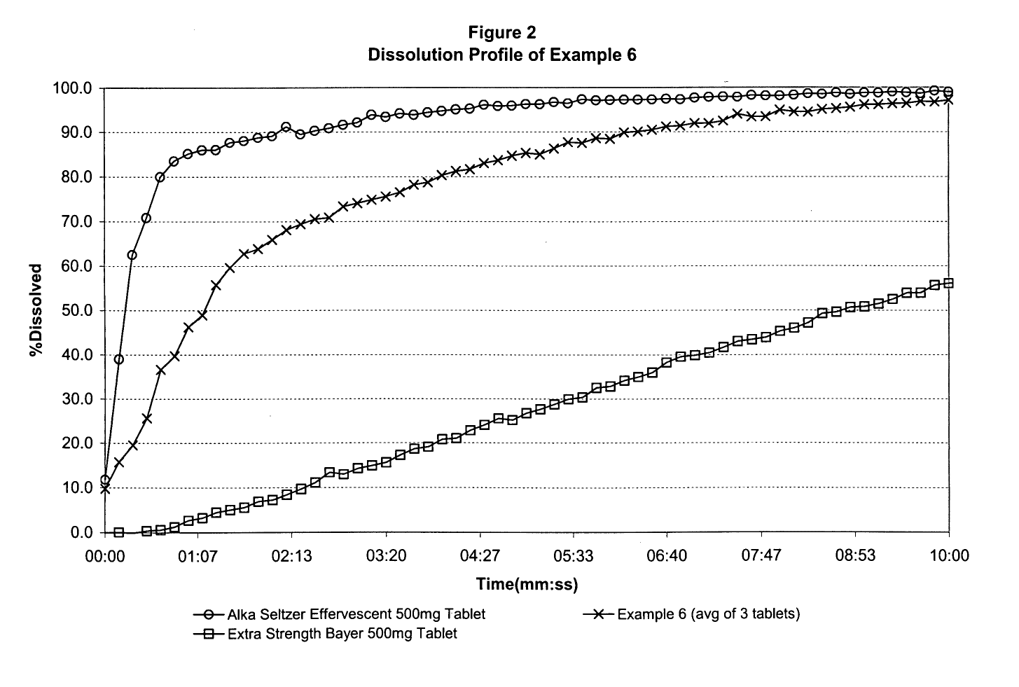

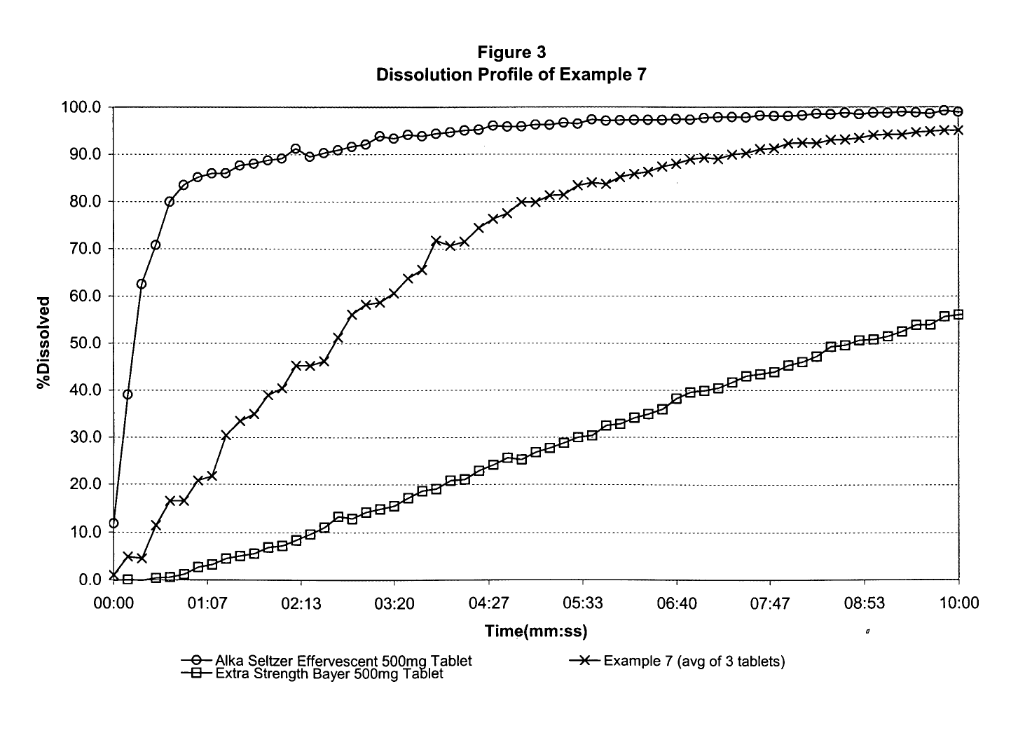

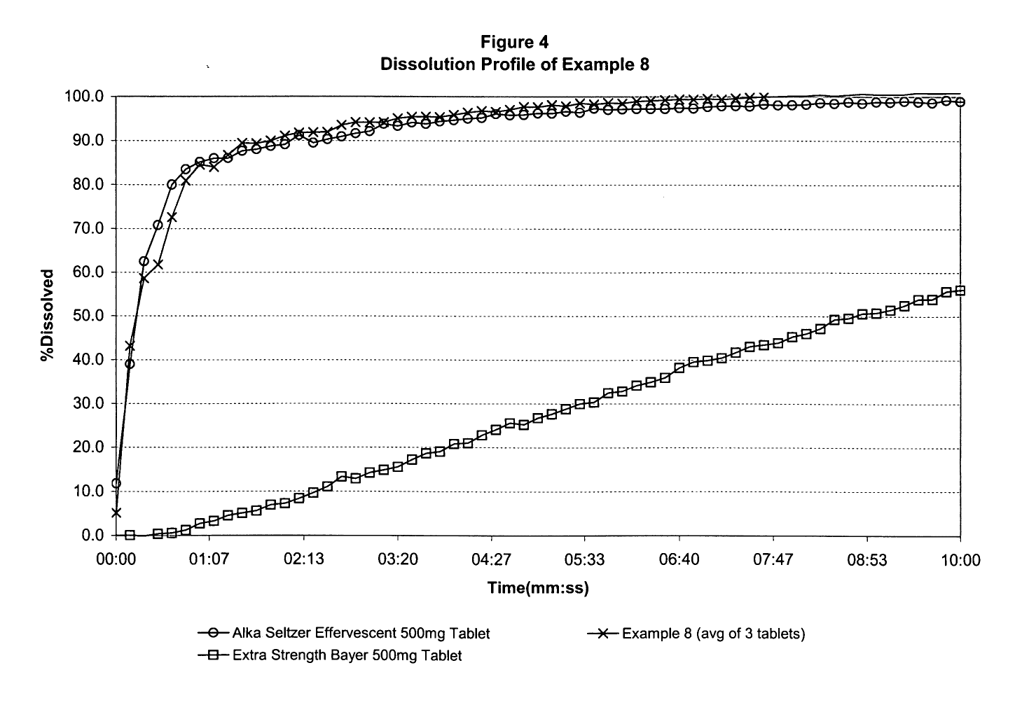

Tablets each prepared in accordance with Examples 5-8 were evaluated for their suitability as effervescent tablets and as swallowable tablets. The dissolution profiles for three tablets of 500 mg tablets of each formulation were separately measured using standard dissolution tests. The sample basket was rotated at 50 rpm in a 500 mL acetate buffered aqueous medium maintained at pH 4.5. The results were compared to dissolution profiles of 500 mg Citrus Flavor Alka-Seltzer® brand effervescent tablets and 500 mg Extra Strength Bayer® brand aspirin tablets, and the averaged results are shown in FIGS. 1-4.

### Examples 13-17

Examples 5-8 were tested against Citrus Flavor Alka-Seltzer® brand 500 mg tablets (identified as “Eff.” in Table 3) and Extra Strength Bayer® 500 mg tablets (identified as “ESB” in Table 3) to evaluate several properties that are shown in Table 3. Table 3 shows (a) the weight ratio of aspirin, carbonate and disintegrant in the examples; (b) the time to complete dissolution of the tablets in 150 ml water measured in minutes and seconds, (c) the residue in the resulting solution on a 1-5 scale, with 1 being no residue and 5 being a large amount of residue; (d) the transparency of the resulting solution on a 1-5 scale, with 1 being completely clear and 5 being very opaque, (e) the taste of the resulting solution on a 1-5 scale with 1 being the lowest; and (f) the average tablet weight of the tested samples in milligrams. The results are reported in increasing time for complete dissolution.

TABLE 3
Effect of Disintegrant

| Sample | Eff. | Ex. 8 | Ex. 6 | Ex. 5 | Ex. 7 | ESB |
|---|---|---|---|---|---|---|
| Wt. Ratio* | n/a | 84/15/1 | 87/12/1 | 87/12/1 | 88/12/0 | n/a |
| Complete | 1:30 | 1:30 | 2:05 | 4:42 | 12:10 | 50 sec** |
| Dissolution | | | | | | |
| Time | | | | | | |
| Residue | 1 | 1.5 | 3 | 2 | 5 | 5 |
| Transp. | 1 | 1.5 | 2 | 2 | 1 | 2.5 |
| Masking | n/a | 2 | 1.5 | 1.5 | 1.5 | n/a |
| Average | 3500 | 594 | 574 | 574 | 568 | 600 |
| Tablet | | | | | | |
| Weight | | | | | | |

*Ratio of Aspirin/carbonate/disintegrant
**disintegrated, then undissolved remainder stayed at bottom

### Examples 18-26

Examples having the formulations set forth in Table 4 were evaluated for disintegration time, taste characteristics of the resulting solution, and residue when used as effervescent tablets. Examples 22, 23, and 24 had a combination of acceptable disintegration times (less than 3 minutes), acceptable taste characteristics and acceptable residue levels.

TABLE 4
Properties of Formulations*

| | | | | | | Dis- | |
|---|---|---|---|---|---|---|---|
| | | Wt % | | | | integration | |
| | ASA | ASA | Na₂CO₃ | Kollidon | Saltiness | Time | Residue |
| Ex. | (mg) | (0-180) | (Ground) | CL-SF | (1: Low) | (min) | (1: Clear) |
| 18 | 325 | 75 | 25 | 0 | 4 | <2 | 1 |
| 19 | 325 | 79 | 20 | 0 | 2 | <3 | 1 |
| 20 | 500 | 75 | 25 | 0 | 5 | <3 | 1 |
| 21 | 500 | 79.5 | 20 | 0.5 | 3 | <3 | 1.5 |
| 22 | 500 | 84.5 | 15 | 0.5 | 2 | <3 | 1.5 |
| 23 | 500 | 84 | 15 | 1 | 2 | <3 | 1.5 |
| 24 | 500 | 87 | 12 | 1 | 1.5 | <3 | 2 |
| 25 | 500 | 89 | 10 | 1 | 1 | <4 | 3 |
| 26 | 500 | 94 | 5 | 1 | 1 | <5 | 4 |

*All tablets were made by carver press.

The results also suggested that formulations having non-micronized bicarbonate are preferred over formulations having micronized bicarbonate, which in turn are preferred over co-micronized aspirin and bicarbonate. Effervescent formulations containing more than about 1% by weight of the super-disintegrant showed adverse transparency effects in the resulting solution when compared with formulations having less than about 1% by weight of the super-disintegrant.

The purpose of the above description is to illustrate some embodiments of the present invention without implying a limitation. It will be apparent to those skilled in the art that various modifications and variations may be made in the apparatus or procedure of the invention without departing from the scope or spirit of the invention.

What is claimed is:

1. A tablet comprising: (a) an active ingredient; and (b) an effervescent couple, wherein said tablet dissolves in water in not more than three minutes and in the mouth in not more than thirty seconds.

2. The tablet of claim 1, wherein said tablet further comprises a super-disintegrant.

3. The tablet of claim 1, wherein said active ingredient is acidic.

4. The tablet of claim 1, wherein said active ingredient is basic.

5. The tablet of claim 4, wherein said active ingredient is aspirin.

6. The tablet of claim 1, wherein the basic member of said effervescent couple is selected from the group consisting of sodium bicarbonate, sodium carbonate, potassium carbonate, potassium bicarbonate, calcium carbonate, magnesium carbonate and mixtures thereof.

7. The tablet of claim 1, wherein one or more of the ingredients of said tablet is micronized to a particle size of less than about 75 microns.

8. A tablet comprising (a) an active ingredient; and (b) one member of an effervescent couple, wherein said tablet dissolves in water in not more than three minutes and in the mouth in not more than thirty seconds and wherein said active ingredient is also the other member of said effervescent couple.

9. The tablet of claim 8, wherein said tablet further comprises a super-disintegrant.

10. The tablet of claim 8, wherein said active ingredient is acidic.

11. The tablet of claim 8, wherein said active ingredient is basic.

12. The tablet of claim 8, wherein said active ingredient is aspirin.

13. The tablet of claim 8, wherein the basic member of said effervescent couple is selected from the group consisting of sodium bicarbonate, sodium carbonate, potassium carbonate, potassium bicarbonate, calcium carbonate, magnesium carbonate and mixtures thereof.

14. The tablet of claim 8, wherein one or more of the ingredients of said tablet is micronized to a particle size of less than about 75 microns.

15. A tablet comprising: (a) aspirin; (b) at least one member selected from the group consisting of sodium bicarbonate, sodium carbonate, potassium carbonate, potassium bicarbonate, calcium carbonate, magnesium carbonate and mixtures thereof; and (c) a super-disintegrant, wherein said tablet dissolves in water in not more than three minutes and in the mouth in not more than thirty seconds, and wherein said tablet is capable of acting as an effervescent tablet, a rapid-dissolve tablet or a rapidly disintegrating swallow tablet.
